SEQUENCE LISTING (3 sequences)

SEQ ID NO: 1 - DNA, 22 bp, Artificial Sequence
  gRNA
aagcataaccttcggcaaaggg

SEQ ID NO: 2 - DNA, 22 bp, Artificial Sequence
  gRNA
gagcagagttctgttgtccggg

SEQ ID NO: 3 - DNA, 22 bp, Artificial Sequence
  gRNA
ccctgactatgatgccgccagc

(19) United States
(12) Patent Application Publication
(10) Pub. No.: US 2019/0192698 A1
(43) Pub. Date: Jun. 27, 2019

(54) A METHOD FOR OBTAINING INDICATOR SIGNALS FROM A CELL

(71) Applicant: TEKNOLOGIAN TUTKIMUSKESKUS VTT OY, Espoo (FI)

(72) Inventors: Eero PUNKKA, Helsinki (FI); Seppo VAINIO, Oulu (FI)

(21) Appl. No.: 16/064,814

(22) PCT Filed: Dec. 22, 2016
(86) PCT No.: PCT/FI2016/050917

§ 371 Date: Jun. 21, 2018

(30) Foreign Application Priority Data
Dec. 23, 2015 (FI) 20156008

(51) Int. Cl. A61K 49/00; C12Q 1/00; C12N 5/071

(52) U.S. Cl. CPC A61K 49/0054; C12N 2506/09; C12N 5/0602; C12Q 1/006

(57) ABSTRACT

The present invention relates to a field of genetically edited cells and furthermore determining indicator signals of genetically edited cells. The invention relates to a method for obtaining indicator signals from a cell, and more particularly to a method for determining a biological state of a cell. Furthermore, the present invention relates to a regenerative cell and use of a regenerative cell or a specific indicator poly-nucleotide for monitoring purposes. Also, a system for carrying out the method of the present invention is included.

This application is the U.S. national phase of International Application No. PCT/FI2016/050917 filed 22 Dec. 2016, which designated the U.S. and claims priority to FI Patent Application No. 20156008 filed 23 Dec. 2015, the entire contents of each of which are hereby incorporated by reference.

### FIELD OF THE INVENTION

The present invention relates to a field of genetically edited cells and furthermore determining indicator signals of genetically edited cells. The invention relates to a method for obtaining indicator signals from a cell, and more particularly to a method for determining a biological state of a cell. Furthermore, the present invention relates to a regenerative cell and use of a regenerative cell or a specific indicator polynucleotide for monitoring purposes. Also, a system for carrying out the method of the present invention is included.

### BACKGROUND OF THE INVENTION

Targeted genetic editing/engineering is a very important tool for e.g. deleting or adding genes, removing or inserting exons or introducing or correcting point mutations. There are several types of genetic editing techniques in the art. As an example, Urnov et al. has corrected mutations of the IL2Ry gene by using ZFNs (Urnov et al. 2005, Nature 435: 646-651). TALLEN or Crisp/cas technology has been utilized e.g. by Hockemeyer et al. and Sander and Joung (Hockemeyer et al. 2012, Nat Biotechnol 29(8): 731-734; Hockemeyer et al. 2009, Nat Biotechnol 27(9): 851-857, Sander and Joung, 2014, Nature Biotechnology 32, 347-355). Furthermore, studies on different cell types having specific targeted genetic modifications have been published within recent years. For example Hockemeyer et al. have described gene targeting for in vitro modification of the genomes of human embryonic stem cells (hESC) and induced pluripotent stem cells (iPSCs) (Hockemeyer et al. 2012, Nat Biotechnol 29(8): 731-734; Hockemeyer et al. 2009, Nat Biotechnol 27(9): 851-857).

Genetic editing of cells leads to a need for detecting the molecular changes caused by said editing. Conventional DNA/RNA/protein sequencing and blotting methods have been utilized for determining changes in specific protein, RNA or DNA contents and various PCR techniques and immunohistochemistry have also been very useful for observing the molecular alterations. Converting the endogenous molecules as biosensors via gene editing has widened up the possibilities for monitoring biochemical processes of cells and has opened up the new era of measuring responses of the biological systems to stimuli.

Still, there exists a great need for more simple, low cost, highly sensitive and optimal methods for determining molecular changes of a cell in the context of vital measure of biological responses of the cellular and tissue units to internal and external stimuli.

### BRIEF DESCRIPTION OF THE INVENTION

An object of the present invention is to provide a simple, very sensitive and specific method for genetically edited cell illustration and thereafter monitoring specific indicator signals and/or changes thereof outside of said cell, e.g. on the skin of a subject. In other words, by the method of the present invention indicator signals produced by modified regenerative multipotent cells (e.g. skin derived regenerative cells) may be monitored on the skin of a subject. The objects of the invention are achieved by a method and arrangements which are characterized by what is stated in the independent claims. The preferred embodiments of the invention are disclosed in the dependent claims.

The invention is based on the realization that cellular processes and changes thereof can be monitored ex vivo when using the method of the present invention. The present invention utilizes non-invasive monitoring of indicator signals produced by bioedited genes, proteins or metabolites of cells in a manner that allows real-time monitoring of a given biological process. The invention is, instead of using primarily a physical, man made technological machine as the primary measuring criteria of bodily functions, to use a cellular measuring capacity for a given biologically relevant process (such as glucose metabolism). The present invention utilizes a combination of method steps, wherein a cell is genetically edited ex vivo or in vitro to indicate specific biological changes that are highlighted by molecules indicating a defined biological process and changes in it. The monitoring of the process that a bioindicator depicts is conducted outside of the cell by the presented invention as well.

The invention is based on the study wherein a polynucleotide sequence encoding an indicator is inserted into DNA of a cell and the polynucleotide of the indicator is expressed together with a specific target polynucleotide. The target polynucleotide to be inserted with the indicator polynucleotide has been selected based on its ability to be expressed in response to the presence or absence of an analyte of interest. In other words the present invention exploits a situation where expression of a specific gene responds to a specific analyte or physical or energetic stimuli. Therefore, changes in the defined responses in a cell to the factors that influence homeostasis can be studied by monitoring the edited signals generated by the indicators. Furthermore, if several different indicator polynucleotides are inserted into DNA of a cell, more than one analyte or stimuli may be monitored at the same time.

The present invention provides a tool for monitoring cells of a specific type (e.g. skin derived regenerative cells).

The present invention solves a problem related to a lack of specific methods for studying molecular changes of a cell by real time monitoring from outside of the cell. Furthermore, the present invention provides tools and conditions for studying molecular changes or state of a cell.

The present invention provides a flexible, simple, low-cost, convenient, efficient, specific, sensitive and reliable method for determining cellular conditions.

The method of the present invention enables further improvements in molecular biology and enables determining how the living systems respond to environment. Furthermore, the present invention helps in understanding changes of specific cells and determining the very specific changes in cells and between them.

The present invention also provides a more personalized method for determining cell biological events of a subject and it is based on the fact that each individual is unique in its genetic make up. Thus the innovation provides also the technology for biomonitoring at a personalized level. The real-time monitoring may continue all the time and anywhere, because the monitoring occurs non-surgically and may take place e.g. on the edge of the skin of a patient. Actually a healthy or diseased patient may easily take care of the monitoring with-out trained professionals to acquire and follow the accumulation of the bioindicator results.

If people are able to continuously monitor their specific physiological states, development of more severe disorders may be diminished and the needs to visit hospitals or health clinics may be avoided. In most optimal cases diseases may be totally prevented due to the biofeedback provided by the invention. Indeed, the present invention enables also foreseeability and early intervention compared to methods used for conventional diagnostics.

The present invention provides a method for overcoming the limitations of laborious, slow and high cost methods for obtaining results, which can be used for planning e.g. therapies, medical treatments, diet, life style, mood or routine way of life factors.

In one aspect, the present invention relates to a method for obtaining indicator signals from a cell, said method comprising

providing regenerative cells obtained from a subject,

modifying the regenerative cell by inserting a polynucleotide sequence encoding an indicator into DNA of the regenerative cell, the polynucleotide sequence encoding the indicator to be expressed together with a target polynucleotide in said regenerative cell,

administering the modified regenerative cell to a subject,

monitoring a signal of the indicator or absence thereof on the skin of the subject, thereby obtaining indicator signals associated with expression of the target polynucleotide.

In one aspect the present invention relates to a method for determining a biological state of a cell, said method comprising

providing regenerative cells obtained from a subject,

modifying the regenerative cell by inserting a polynucleotide sequence encoding an indicator into DNA of a regenerative cell, the polynucleotide sequence encoding the indicator to be expressed together with a target polynucleotide in said regenerative cell,

administering the modified regenerative cell to a subject,

monitoring a signal of the indicator associated with expression of the target polynucleotide or absence of said indicator signal on the skin of the subject, and

determining a biological state of the cell defined by expression of the target polynucleotide.

Furthermore, in one aspect, the present invention relates to use of a regenerative cell obtained from a subject and modified to comprise an inserted polynucleotide sequence of an indicator to be expressed together with a target polynucleotide for monitoring an indicator signal associated with expression of the target polynucleotide, wherein monitoring is carried out on the skin of a subject.

Furthermore, in one aspect, the present invention relates to use of a skin derived regenerative cell obtained from a subject and modified to comprise an inserted polynucleotide sequence of an indicator to be expressed together with a target polynucleotide for monitoring an indicator signal associated with expression of the target polynucleotide from outside of the cell.

Furthermore, in one aspect, the present invention relates to a regenerative cell obtained from a subject and comprising an inserted polynucleotide sequence encoding an indicator to be expressed together with a target polynucleotide optionally for measuring the indicator signal associated with expression of the target polynucleotide on the skin of a subject.

Still in a further aspect the present invention relates to a genetically modified regenerative cell comprising an inserted polynucleotide sequence encoding an indicator to be expressed together with a target polynucleotide for use in obtaining an indicator signal to be monitored on the skin of a subject.

Still, in one aspect, the present invention relates to use of a polynucleotide encoding an indicator, which polynucleotide is inserted into a target polynucleotide in a regenerative cell and which polynucleotide is to be expressed together with the target polynucleotide, for determining an analyte of the cell, wherein the analyte is able to control expression of the target polynucleotide.

Still, in a further aspect the present invention relates to a method for determining a biological state of a subject in need thereof, said method comprising

obtaining regenerative cells from a subject,

modifying the regenerative cell by inserting a polynucleotide sequence encoding an indicator into DNA of a regenerative cell, the polynucleotide sequence encoding the indicator to be expressed together with a target polynucleotide in said regenerative cell,

administering the modified regenerative cell to a subject,

monitoring a signal of the indicator associated with expression of the target polynucleotide or absence of said indicator signal on the skin of the subject, and

determining a biological state of the subject defined by expression of the target polynucleotide.

Still further, in one aspect the present invention relates to a method for obtaining indicator signals from a cell, said method comprising

obtaining regenerative cells from a subject,

modifying a regenerative cell by inserting a polynucleotide sequence encoding an indicator into DNA of the regenerative cell, the polynucleotide sequence encoding the indicator to be expressed together with a target polynucleotide in said regenerative cell,

administering the modified regenerative cell to a subject, and

monitoring a signal of the indicator or absence thereof on the skin of the subject, thereby obtaining indicator signals associated with expression of the target polynucleotide.

Still, in a further aspect the present invention relates to a system comprising

means for modifying the regenerative cell by inserting a polynucleotide sequence encoding an indicator into DNA of the regenerative cell, the polynucleotide sequence encoding the indicator to be expressed together with a target polynucleotide in said regenerative cell,

means for applying the modified cells on the skin of a subject or into a subject, and

means for monitoring a signal of the indicator or absence thereof on the skin of the subject.

### BRIEF DESCRIPTION OF THE DRAWINGS

In the following the invention will be described in greater detail by means of preferred embodiments with reference to the attached [accompanying] drawings, in which

FIG. 1 shows xenograft of skin stem cells using silicon chamber.

FIG. 2 shows GFP skin graft (the whole skin) on the back of WT recipient mouse.

FIG. 3 shows fluorescence spectra of both GFP and wild type (WT) skin of a mouse.

FIG. 4 shows image and spectrum of green fluorescence protein (GFP) mouse skin taken with hyperspectral camera (light source, filter and a multicolour image sensor) and OU Olympus microscope. FIG. 4 reveals that the method of the present invention is suitable for obtaining indicator signals on the skin.

FIG. 5 shows image and spectrum of indicator signals obtained from skin by the method of the present invention. Clear fluorescence signal from area with dye was compared to area without fluorescence dye (A). FIG. 5 reveals that the method of the present invention is suitable for obtaining indicator signals on the skin of a subject. The image has been taken using OU Olympus microscope and a camera to monitor GFP-derived fluorescence (B).

FIG. 6 shows an example of possible measurement setup for the present invention.

FIG. 7 shows an example of a measurement built into a wrist instrument.

FIG. 8 shows schematics of the setup A) and B) suitable for the present invention. A) The means for monitoring comprised optomechanical components, lenses filters and other parts. Led or white-light source was used with an appropriate filter to illuminate the fluorescing target. Camera was used for detection. Fluorescence (e.g. glucose-induced) was measured with this setup and necessary detection limits and intensity variation scale were tested. B) LED was selected for appropriate wavelength and the illumination area was defined. Small sensor, CCD-camera/row detector or single detector was selected, necessary electronics circuits and the mechanical mount for the reader were obtained. The signal acquisition was based on PC data acquisition cards, and the signal processing was made with any appropriate programming language (e.g. Labview).

FIG. 9 shows quantitative PCR (QPCR) and RNA sequencing results for different polypeptides coding for (A) kalkrein 6, (B) Sprr1b and (C) Pyhin 1.

FIG. 10 shows skin tissue proteins significantly changed (minimum 1.5 fold) by glucose injection.

### DETAILED DESCRIPTION OF THE INVENTION

In this invention specific indicators associated with cellular processes can be monitored in real time from regenerative cells. The idea is to use indicators in a target cell and let the indicator transmit the given biological response to a skin-attached device that converts the signal e.g. to numeric values for monitoring and control purpose. The cell-level indicator becomes activated as a response of a given biological or environmental stimulus (i.e. Specific analyte or energy causes expression of a target polynucleotide and at the same time also expression of an indicator polynucleotide) and downregulated or inactivated completely when the stimulus ends. The indicator is targeted to a regenerative cell to generate the cell as a sensitive and vital biosensor. The regenerative cell becomes as part of cells of the donor or another subject. In a specific embodiment the modified regenerative cell becomes as part of a basal cell layer that renews the skin, thus acting as an indicator and sensor throughout the life time of the subject or patient. The cell may be eradicated also via small molecule induced mechanism. The biological indicator signal within the gene edited regenerating cell is converted to a measurable signal serving as a real-type and vital biosensor in biomonitoring.

By the present invention the sensory mechanism is embedded into one or more living cells and it is possible to determine a biological state of the cell(s) in a dynamic fashion. As used herein “a biological state” refers to any state of a cell, which is defined by the amount, presence, absence or activity of a specific analyte or process that is relevant in maintenance or loss of homeostasis.

As used herein, the term “or” has the meaning of both “and” and “or” (i.e. “and/or”). Furthermore, the meaning of a singular noun includes that of a plural noun and thus a singular term, unless otherwise specified, may also carry the meaning of its plural form. In other words, the term “a” or “an” may mean one or more.

### Cells

A cell to be genetically edited according to the present invention is a regenerative cell such as a somatic regenerative cell. As used herein “a regenerative cell” refers to a cell, which is able to self-renew and differentiate. In one embodiment of the invention a regenerative cell is selected from the group consisting of somatic stem cells (i.e. cells, which are able to maintain and repair the tissue in which they are found) e.g. cells of the bone marrow, adipose tissue, blood, epithelium, endothelium and/or mesenchyme. A regenerative cell of the invention includes but is not limited to a multipotent stem cells (which can differentiate into a number of cell types, but only those of a closely related family of cells), oligopotent stem cells (which can differentiate into only a few cell types, such as lymphoid or myeloid stem cells) and/or unipotent cells (which can produce only one cell type, their own, but have the property of self-renewal, which distinguishes them from non-stem cells). Regenerative cells of the present invention may also include pluripotent adult stem cells, which are rare and generally small in number, and present e.g. in the bone marrow.

As used herein, the terms “genetically edited cells” and “genetically modified cells” are interchangeable.

In one embodiment of the invention the regenerative cell is selected from the group consisting of skin derived regenerative cells, blood derived regenerative cells and iPS cells.

In one embodiment of the invention the regenerative cell is an induced pluripotent stem cell (iPSC) e.g. in their differentiated progenitors. As used herein, the term “induced pluripotent stem cells” (iPSCs) refers to pluripotent stem cells generated from differentiated cells, typically from adult somatic cells such as fibroblasts by developmental reprogramming. Such cells have been described e.g. in WO 2008/151058 and US 2008/076176.

In one embodiment of the invention the regenerative cell is an embryonic stem cell or an embryonic stem cell derived cell. Embryonic stem cells (ESCs) are pluripotent cells having the ability to differentiate into a wide variety of different cell types, such as endothelial cells. Methods of obtaining embryonic stem cells are readily available in the art. In addition, WO 2007/130664 discloses a promising new approach, termed blastomere biopsy, for obtaining human embryonic stem cells without damaging the donor embryo.

In another embodiment of the invention the regenerative cell is a skin derived regenerative cell i.e. a regenerative cell obtained from a skin. As used herein “a skin derived regenerative cell” refers to a cell, which is obtained from a skin and is able to self-renew and differentiate into multiple lineages. In a more specific embodiment a skin derived regenerative cell is from the basal layer of the skin or from the sweat gland or hair follicles. The skin constantly renews itself throughout adult life, and the follicles undergo a perpetual cycle of growth and degeneration. Stem cells residing in the epidermis and hair follicle ensure the maintenance of adult skin homeostasis and hair regeneration, but they also participate in the repair of the epidermis after injuries.

In an adult, different types of stem cells function to replenish various cell types in skin as it undergoes normal homeostasis or wound repair. Some stem cells (e.g., melanoblasts and epidermal stem cells) reside within the skin itself. Mature epidermis is a stratified squamous epithelium whose outermost layer is the skin surface. Only the innermost (basal) layer is mitotically active. The basal layer produces, secretes, and assembles an extracellular matrix, which constitutes much of the underlying basement membrane that separates the epidermis from the dermis. As cells leave the basal layer and move outward toward the skin surface, they withdraw from the cell cycle and execute a terminal differentiation program. In the early stages of producing spinous and granular layers, the program remains transcriptionally active. However, it culminates in the production of dead flattened cells of the cornified layer (squames) that are sloughed from the skin surface, continually being replaced by inner cells moving outward. (Blanpain C and Fuchs E, 2006, Annu Rev Cell Dev Biol 22: 339-373).

In a specific embodiment of the invention the skin derived regenerative cell is an epidermal stem cell or a stem cell of the hair follicle. These stem cells possess two essential features common to all stem cells. They are able to self-renew for extended periods of time, and they differentiate into multiple lineages derived from their tissue origin (Weissman IL et al. 2001, Annu Rev Cell Dev Biol. 17:387-403, Blanpain C and Fuchs E, 2006, Annu Rev Cell Dev Biol 22: 339-373). In another specific embodiment of the invention the skin derived regenerative cell is a keratinocyte. Keratinocytes are the predominant cell type in the epidermis, the outermost layer of the skin, constituting 90% of the cells found there. Those keratinocytes found in the basal layer of the skin are sometimes referred to as “basal cells” or “basal keratinocytes”.

In one embodiment of the invention the regenerative cell is a blood derived regenerative cell i.e. a regenerative cell obtained from blood.

In a specific embodiment a cell to be modified and monitored is a primary regenerative cell (e.g. a primary epidermal stem cell). As used herein “a primary cell” refers to a cell, which has not undergone many population doublings and thus closely represents the physiological state of cells in vivo and generates relevant data representing living systems. Primary cells are cells taken directly from a living tissue and established for culture in vitro. Primary cells are not e.g. tumor or immortalized cell lines.

In another embodiment of the invention a cell to be modified and monitored is not a primary regenerative cell.

In a specific embodiment the method of the invention further comprises a step of culturing regenerative cells. The cells may be cultured either in ex vivo or in vitro culture conditions.

Cells used in the method of the invention are obtained from a subject. The subject may be selected from a human or animal subject. In a specific embodiment, the cells for genetic modification are either human or animal cells. Preferable animal cells include but are not limited to cells of nonhuman primates such as chimpanzees and other apes and monkey species; birds; farm animals such as poultry, cattle, sheep, pigs, goats, and horses; domestic mammals such as cagebirds, dogs and cats; laboratory animals including rodents such as mice, rats, rabbits, guinea pigs, and the like. In a very specific embodiment, the subject is a human (e.g. a child (age from 0 until 18 years) or an adult (age starting from 18 years)). The subject may be selected from a human or animal subject with the proviso that if human embryonic stem (hES) cells are used, the method does not include the destruction of human embryos.

In one embodiment of the invention a regenerative cell obtained from a subject comprises an inserted polynucleotide encoding an indicator to be expressed together with a target polynucleotide and furthermore an indicator signal. In a specific embodiment the cell is for monitoring an indicator signal by measuring the signal associated with expression of the target polynucleotide from outside of the cell or on the skin of a subject.

In some embodiments, a regenerative cell to be modified and monitored may be obtained from a subject who is heterologous to the recipient subject to be administered with the cell. However, in preferred embodiments, the regenerative cell to be modified and monitored is obtained from a subject who is homologous (i.e. allogenic), more preferably autogenic, to the recipient subject to be administered with the cell. Accordingly, suitable subjects to be administered with the modified regenerative cell include those disclosed above in connection with suitable animal cells to be modified and monitored.

### Polynucleotide Targeting

Polynucleotide targeting (i.e. gene targeting) uses homologous recombination to target desired changes to a specific endogenous polypeptide. The success of polynucleotide targeting can be enhanced with the use of engineered nucleases such as zinc finger nucleases, engineered homing endonucleases, transcription activator-like effector nuclease or CRISPR. Engineered nucleases can also introduce mutations at endogenous genes that generate a gene knockout.

In the present invention polynucleotide targeting is used to insert an indicator polynucleotide into a target polynucleotide. Polynucleotide targeting can be permanent or conditional. In a specific embodiment of the invention polynucleotide targeting is permanent. Polynucleotide targeting requires the creation of a specific vector for each target polynucleotide of interest. However, the vector can be used for any indicator polynucleotide, regardless of transcriptional activity or size. The term “vector” refers to a nucleic acid compound and/or composition that transduces a cell, thereby causing the cell to express polynucleotides and/or polypeptides other than those native to the cell, or in a manner not native to the cell. In general, a targeting construct made out of DNA is generated in bacteria. A construct typically contains part of the polynucleotide to be targeted and an indicator polynucleotide, optionally also a selectable marker. In order to target specific polynucleotides to DNA of ex vivo or in vitro cells a polynucleotide targeting construct is inserted into a cell in culture. Cells with the correct insertion may be selected based on the marker.

Polynucleotide targeting may be carried out by any methods or techniques well known in the art. Methods for genetic targeting are described in various practical manuals describing laboratory molecular techniques. A person skilled in the art knows when and how to employ these methods.

Polynucleotide targeting of the invention may be carried out by using artificially engineered nucleases. The nucleases create specific double-stranded break at desired locations in the genome, and harness the cell's endogenous mechanisms to repair the induced break by natural processes of homologous recombination and nonhomologous end-joining. Nucleases suitable for the present invention include but are not limited to, Zinc finger nucleases, Transcription Activator-Like Effector Nucleases (TALENs), the CRISPR/Cas system, and engineered meganuclease re-engineered homing endonucleases. In a specific embodiment of the invention insertion of a polynucleotide sequence encoding the indicator is carried out by using site-specific nucleases. As used herein “site-specific nucleases” refers to nucleases, which create double-stranded breaks at desired locations. In another specific embodiment of the invention insertion of a polynucleotide sequence encoding the indicator is carried out by zinc finger nuclease (ZFN), transcription activation-like effector nuclease (TALEN) mediated genome editing or CRISPR/Cas system. ZFNs are artificial restriction enzymes generated by fusing a zinc finger DNA-binding domain to a DNA-cleavage domain. TALENs are artificial restriction enzymes generated by fusing a TAL effector DNA binding domain to a DNA cleavage domain. ZFNs and TALENs can be quickly engineered to bind practically any desired DNA sequence because their DNA binding domains can be designed to target desired DNA sequences and this enables nucleases to target unique sequences even within complex genomes. Specificity of methods using ZFNs and TALENs is due to DNA binding domains, which direct DNA cleavages to the neighboring sequences. ZFN and TALEN techniques are described in various practical manuals describing laboratory molecular techniques and for example in the articles of Hockemeyer et al. (Hockemeyer et al. 2012, Nat Biotechnol 29(8): 731-734; Hockemeyer et al. 2009, Nat Biotechnol 27(9): 851-857). CRISPR/Cas system has been described e.g. in the article of Sander and Joung (2014, Nature Biotechnology 32, 347-355). A person skilled in the art knows when and how to employ these methods.

Optionally, screening of the polynucleotide targeting may be performed by sequencing, PCR or Southern analysis to confirm that the desired genetic insertion has taken place or to identify the point of integration of an indicator polynucleotide.

### Target Polynucleotide

In the present invention a polynucleotide sequence encoding an indicator is inserted into a specific place of the DNA of a cell and the polynucleotide of the indicator is expressed together with a target indicator gene of interest. Indeed, the indicator polynucleotide is inserted into defined and designed region(s) in the DNA. The site of the DNA, wherein the polynucleotide is inserted may be within the target polynucleotide sequence to be expressed or outside of the target polynucleotide sequence, e.g. in the same cluster (gene cluster) with the target polynucleotide. A gene cluster comprises at least two polynucleotide sequences encoding polypeptides, which polynucleotide sequences are usually grouped together and expressed together. Therefore a person skilled in the art understands that according to the present invention the indicator polynucleotide is expressed together or at the same time with the target polynucleotide. This enables follow up of the expression of the target polynucleotide.

Insertion of an indicator polynucleotide into the DNA does not have negative effect on the function of a cell. For example, if the indicator polynucleotide is inserted into the target polynucleotide to be expressed and a fusion polypeptide is formed, the function of the fusion polypeptide is comparable to the function of the unmodified polypeptide. Hence there is no need to remove host DNA sequences and the editing represents the very minimal to the genome and is confirmed.

In a specific embodiment of the invention a polynucleotide sequence encoding an indicator is inserted into a target polynucleotide. In a very specific embodiment of the invention a polynucleotide sequence encoding an indicator is inserted into a 3′ end of a target polynucleotide. A site of the target polynucleotide for targeting may be selected from any site within a coding polynucleotide sequence or any noncoding or regulatory sequence. It is well known to a person skilled in the art that suitable sites for insertion depend on the specific target polynucleotide in question.

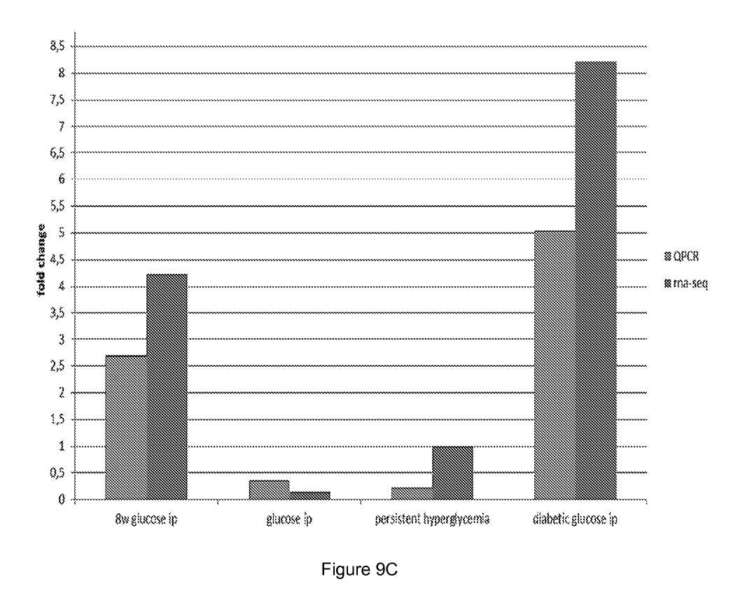

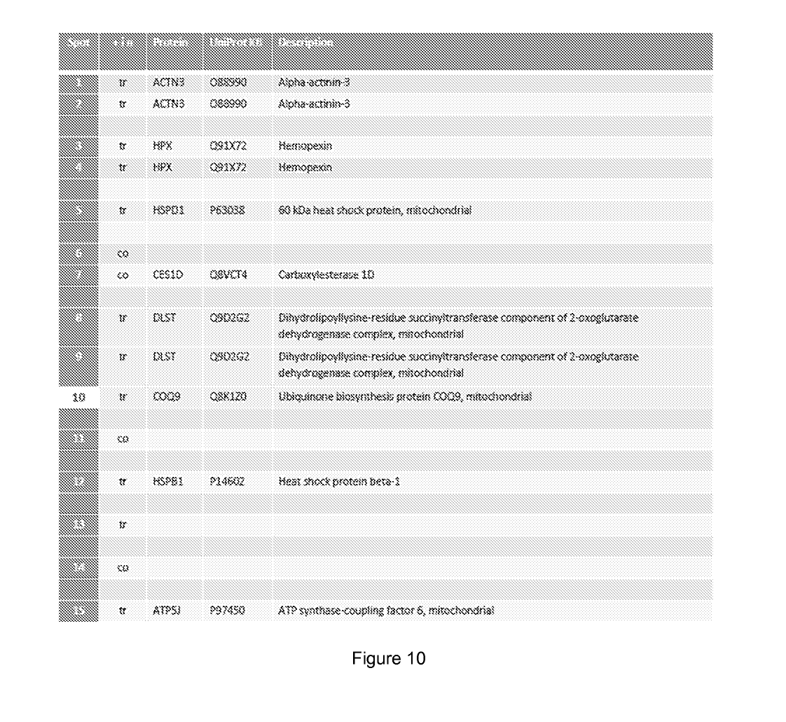

A target polynucleotide to be edited as a bioindicator/reporter may be any polynucleotide whose expression may be affected by an analyte. In a specific embodiment of the invention the target polynucleotide is selected from the group consisting of polynucleotides encoding glucose responsive polypeptides, growth factors, mitochondrial enzymes, hormone responsive polypeptides, stress responsive polypeptides, polypeptides of the central or peripheral nervous system function, alcohol or drug responsive polypeptides, polypeptides used in immunological monitoring of disease development, polypeptides revealing changes in physical forces such as pressure or stretching, and polypeptides expressed by physical load in exercise or pathogen infections. Any of processes related to expression of the above mentioned target polynucleotides may be monitored by the present invention based in the identified biomarker that depicts these specific biological processes in homeostasis and deviation from in normal physiological conditions or in disease. The examples of suitable target polynucleotides for the molecular circuits that have been identified include but are not limited to specific enzymes encoding genes that are regulated by the analyte, genes that are targeted by toxics such as ethanol, genes encoding extracellular matrix and enzymes and repair factors that are involved in muscle recovery from exercise load, and genes encoding immunological factors that trigger innate immunity of humoral responses in cells. Polynucleotides encoding glucose responsive polypeptides suitable for the present invention may be selected from, but are not limited to, lists presented in Table 1, Table 2 or Table 3 of Example 1, in FIG. 9 or in FIG. 10 or any combination thereof. As an example, if a target polynucleotide is a glucose responsive polypeptide, then the present invention allows a specific method and tools for exact, reliable and safe ways to assay how the cells responds to glucose. As an example genes mentioned e.g. in Tables 1, 2 or 3 in Example 1, FIG. 9 or FIG. 10 have been induced in vivo in a model organism by glucose and the expression is reduced in the absence of glucose. The present invention as it provides a new way to monitor and measure physiological functions represent also a platform to identify novel analytes and response mechanisms in cells and the tissue made by the regenerative cells.

As used herein “polynucleotide” refers to any polynucleotide, such as single or double-stranded DNA (genomic DNA or cDNA), comprising a nucleic acid sequence encoding a polypeptide in question or a conservative sequence variant thereof. In connection with polynucleotides, the term “conservative sequence variant” refers to nucleotide sequence modifications, which do not significantly alter biological properties of the encoded polypeptide. Conservative nucleotide sequence variants include variants arising from the degeneration of the genetic code and from silent mutations. Nucleotide substitutions, deletions and additions are also contemplated. The term “variant” as used herein refers to a sequence having minor changes in the amino acid or nucleic acid sequence as compared to a given sequence. Such a variant may occur naturally e.g. as an allelic variant, or it may be generated by mutagenesis or other gene modification.

In addition to genetic modification by inserting a polynucleotide sequence encoding an indicator into DNA of a cell, the cell of the present invention may also comprise other genetic modifications. These genetic modifications include any genetic modifications e.g. insertions, deletions or disruptions of one or more genes or a fragment(s) thereof or insertions, deletions or disruptions of one or more nucleotides, or addition of plasmids. As used herein “disruption” refers to insertion of one or several nucleotides into the gene resulting in lack of the corresponding protein or presence of non-functional proteins or protein with lowered activity. Other genetic modifications may be selected from one or several modifications causing down regulation and/or over-expression of a polynucleotide or not affecting the expression of a polynucleotide. As used herein “over-expression” refers to excessive expression of a polynucleotide by producing more products (e.g. polypeptide) than an unmodified cell. For example one or more copies of a polynucleotide or polypeptides may be transformed to a cell for overexpression. The term also encompasses embodiments, where a promoter or promoter region has been modified or a promoter not naturally present in the cell has been inserted to allow the over-expression of the polypeptide. Also, epigenetic modifications such as DNA methylation and histone modifications are included in “genetic modifications”.

In a specific embodiment of the invention no other genetic modifications than an insertion of a polynucleotide sequence encoding an indicator are carried out in a cell to be monitored.

In some embodiments, the target polynucleotide sequence e.g. encoding a polypeptide presented in Table 1, 2 or 3 in Example 1 or in FIG. 9 or 10, may comprise a polynucleotide sequence, which is derivable from public nucleotide sequence databases, or a polynucleotide sequence having at least 70%, 75%, 80%, 85%, 90%, 95%, 96%, 97%, 98%, 99% or 100% nucleic acid sequence identity therewith.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of identity percentage between two sequences can be accomplished using mathematical algorithms available in the art. This applies to both amino acid and nucleic acid sequences.

Sequence identity may be determined for example by using BLAST (Basic Local Alignment Search Tools) or FASTA (FAST-All). In the searches, setting parameters “gap penalties” and “matrix” are typically selected as default.

In one specific embodiment of the invention the indicator polynucleotide is GFP and the target polynucleotide is a polynucleotide encoding a glucose responsive polypeptides, growth factors, mitochondrial enzymes, hormone responsive polypeptides, stress responsive polypeptides, polypeptides of the central or peripheral nervous system function, alcohol or drug responsive polypeptides, polypeptides used in immunological monitoring of disease development, polypeptides revealing changes in physical forces such as pressure or stretching, and polypeptides expressed by physical load in exercise or pathogen infections or any one presented in the list of Table 1, Table 2, Table 3, FIG. 9 or FIG. 10, or any combination thereof.

In a specific embodiment of the invention, the expression of the target polynucleotide is affected by an analyte. As used herein “expression is affected by an analyte” refers to any situation, wherein an analyte is able to control e.g. by starting, increasing, decreasing or stopping expression of a polynucleotide, i.e. expression of the target polynucleotide responds to an analyte.

As used herein “analyte” refers to a molecule, substance or chemical constituent that is of interest in an analytical procedure and has influence (direct or indirect) on expression of the target polynucleotide. “Direct influence” refers to a situation wherein an analyte itself influences expression e.g. by binding to positions of a polynucleotide controlling the expression, whereas “indirect influence” refers to a situation wherein an analyte influences e.g. another analyte of expression of any other polypeptide but not the target polypeptide in question, and by said another analyte or expression influences expression of the target polynucleotide. In a specific embodiment an analyte can be selected from the group consisting of secreted nano- and microvesicles (also called collectively the exosomes, lipid capsulate nano and microscale cell secreted vesicles), bacterial and viral induced toxins, cholesterol derived lipophilic and non-lipophillic hormones and their derivatives, a polynucleotide (e.g. DNA, cDNA, mRNA, siRNA, noncoding RNA, enhancer RNA, free RNA), polypeptide (e.g. a growth factor binding polynucleotide), sugar (e.g. glucose, galactose, lactate), fatty acid, lipid, glycoproteins, metabolite products, electrolyte (e.g. Cl, K, Na, CO₂) and spectral frequencies of physical stimuli. Some examples of analytes which may be detected by the method of the present invention include but are not limited to glucose, insulin, endocrine, paracrine or autocrine hormones, biomarkers and/or pharmaceutical agents. In a specific embodiment, the analyte is a disease-related biomarker. Thus, the invention may involve monitoring biomarkers related to diseases (e.g. diabetes, cardiac disease, cancer, Alzheimer's disease, drug/alcohol/addictions, pathogen infection, immunological monitoring of recovery and host transplant compatibility monitoring, etc).

In a specific embodiment, the analyte is glucose and the target polynucleotide is a polynucleotide comprising a glucose response element (e.g. polynucleotide encoding insulin or glucagon receptor or any of the polypeptides listed in Table 1, 2 or 3 in Example 1, FIG. 9 or FIG. 10). Glucose detection is currently done from a blood sample. However, a specific embodiment of the present invention provides a method for indirectly but at the same time efficiently and specifically following up the glucose level or any other analyte from the skin of a subject.

### Indicator

After a polynucleotide sequence encoding an indicator has been inserted into DNA, it is expressed in a cell together with a target polynucleotide. The indicator polynucleotide sequence encodes a polypeptide or any fragment thereof. The indicator polynucleotide may be natural (e.g. isolated) or may also be generated by taking use of artificial, man synthesized or chemically modified non-natural nucleotides, or the engineered transfer RNA. The indicator polypeptide may emit light as a response to UV exposure such as the Green Fluorescent Protein and the GFP modifications, the lusiferase reporter that generates naturally photons in the visible range, domains of proteins that can interact with aptamers of FRET compounds, the optogenetically active polypeptides that react to visible light frequencies, polypeptides that react to electromagnetic spectral frequencies such as radiofrequencies, myc, flag or halotagged peptides, polypeptides that can be diagnosed by the skin surface located reader in the visible range of frequencies (for example brown, blue, red, green colours generated by melanine producing enzymes), the genes that are derived from the organism such as genes encoding for biological pigment of the host eye or adrenal gland chromophores. In one embodiment of the invention the indicator is selected from the group consisting of fluorescent proteins, a green fluorescence protein (GFP), GFP derivative, photoprotein (e.g. firefly luciferin protein), mCherry, yellow fluorescent protein, tomato red protein, lusiferase reporter, FRET donor and/or acceptor protein, aptamer polynucleotide and/or aptamer polypeptide, myc tag, flag tag, halo tag, biotin/avidin tags and their modifications, unnatural bases and transfer RNA and amino acid based tagging, the polypeptides that serve as electricity indicators and those genes encoding for the pigments of body such as the melanin and eye color pigments.

GFP is a polypeptide composed of 238 amino acid residues and exhibiting bright green fluorescence when exposed to light in the blue to ultraviolet range. GFP refers also to any GFP homologue. As used herein “GFP derivative” refers to a polypeptide comprising amino acid substitutions, deletions or insertions compared to GFP, but still comprising a function in substantially the same manner as the GFP, in particular it retains its capability to exhibit bright green fluorescence when exposed to light in the blue to ultraviolet range. E.g. fusion proteins are within the scope of “a GFP derivative”. In cell and molecular biology, the GFP gene is frequently used as a reporter of expression. The polynucleotide sequence encoding GFP may be introduced into cells and maintained in the genome. As an example, GFP is utilized in a method of article Hockemeyer et al. (Hockemeyer et al. 2009, Nat Biotechnol 27(9): 851-857. The wild type GFP polynucleotide and amino acid sequences are accessible from public sequence databases and GFPs are also commercially available.

Any light emitting polypeptides may be utilized in the present invention. Photoproteins generate light when oxidized and they are commonly used in bioluminescence.

Fluorescence Resonance Energy Transfer (FRET) is the non-radiative transfer of energy from an excited fluorophore (donor) to another fluorophore (acceptor). Exciting the donor and then monitoring the relative donor and acceptor emissions, either sequentially or simultaneously, makes it possible to determine when FRET has occurred. Detection of FRET can be used to quantify when and where two or more biomolecules interact. FRET method has been well described in the art.

Aptamers are oligonucleotide or peptide molecules that bind to a specific target molecule. Aptamers are usually created by selecting them from a large random sequence pool. Aptamers can be combined with ribozymes to self-cleave in the presence of their target molecule. DNA, RNA or nucleic acid analogue aptamers consist of short strands of polynucleotides. Peptide aptamers consist of a short variable polypeptide domain attached at both ends to a protein scaffold. As used herein “protein scaffold” refers to a polypeptide interacting and/or binding with multiple other polypeptides of a signaling pathway, tethering them into complexes. Aptamer methods have been well described in the art and are well known to a person skilled in the art.

GFP, photoprotein, FRET and aptamer techniques are also described in various practical manuals describing laboratory molecular techniques. A person skilled in the art knows when and how to employ these methods.

Optionally and depending on the indicator polynucleotide used, the indicator polynucleotide may be (further) marked or labelled with any label or labelling technique well-known to a person skilled in the art. These labels may be able to give a signal of the indicator. Labelling methods are described in various practical manuals describing laboratory molecular techniques. A person skilled in the art knows when and how to employ these methods. Suitable labels of the indicator include but are not limited to avidin and biotin system or click chemistry based binding of tags. In a specific embodiment of the invention, the indicator has a measurable signal and no further labels are needed. In another specific embodiment the indicator polynucleotide may be edited in a way that the encoded polypeptide is able to bind any agent or molecule, which can be detected by any suitable means.

Monitoring the indicator or signal thereof enables tracking of changes in the expression of the target polynucleotide. Indicator signal of the expressed indicator polynucleotide associates with expression of the target polynucleotide. As used herein “associates with expression of the target polynucleotide” refers to any correlation between the presence, absence, relative abundance or intensity of an indicator signal and expression of the target polynucleotide. Furthermore, the presence, absence or relative abundance of specific analytes in a cell or cells associates with expression of the target polynucleotide and also with expression of the polynucleotide sequence encoding an indicator. Therefore, for example the concentration of an analyte may be determined from outside of the cell(s) by monitoring of the indicator signal.

In a specific embodiment of the invention the indicator forms a fusion polypeptide together with the target polypeptide. In this case the indicator polypeptide is degraded together with the target polypeptide of the fusion polypeptide in the same protein degradation cycle.

In some embodiments, the indicator polynucleotide sequence may comprise a polynucleotide sequence, which may be derived from public nucleotide sequence data bases (such as NIH, EMBL) or a polynucleotide sequence having at least 70%, 75%, 80%, 85%, 90%, 95%, 96%, 97%, 98%, 99% or 100% nucleic acid sequence identity therewith.

In a specific embodiment of the invention the target polynucleotide is any one of those listed in Table 1, 2 or 4 in Example 1, FIG. 9 or FIG. 10, the indicator polynucleotide is GFP and the analyte is glucose.

In one embodiment of the invention genetic modification of a cell used in the present invention does not have any other effect on the function of the cell when compared to an unmodified cell except transcription of the indicator polynucleotide as well as production of the indicator signal. The indicators are non-physiologically functional components, inert in homeostasis control. Based on this several bioindicators may be inserted simultaneously by editing to the cell to be used in biomonitoring. In another embodiment further genetic modifications may be included in the method of the present invention or in products obtained by utilizing said method.

Monitoring the Indicator Signal

Cells genetically modified according to the present invention may be selected from a group of cells including both genetically modified and unmodified cells. According to the present invention the genetically modified cells are administered to a subject. Any administration method may be utilized in the present invention and in one embodiment suitable routes of administration include, but are not limited to, parenteral delivery (e.g. intravenous injection), enteral delivery (e.g. orally), local administration, topical administration (e.g. dermally or transdermally), as known to a person skilled in the art. In a very specific embodiment the genetically modified cells are administered to the basal cell layer or epidermis of the skin that is able to renew the skin.

In one embodiment of the invention the genetically modified regenerative cells or any composition comprising said modified cells are applied on the skin of a subject. In this specific embodiment the modified cells enter the renewing portion of the skin and thus become part of the regenerative cells of the skin and therefore also homeostasis of the skin. In specific the cell that contains the gene edited indicator cell that has been confirmed by means of molecular biological technology is then introduced to the specific cell layers that are in charge of renewing the skin. The alternative for this is that the indicator cell stays episomal. This means that the cell is not integrated to the person but that the cell is inserted, e.g. with a nano needle, to sweat gland gavity. Thus by this way the cell is exposed to the body fluids including the blood derived constituents to and the analytes there in. Moreover the third way to introduce the host derived bio/gene editied indicator cell to the skin is to use it to generate a skin reconstitute by introducing it as part of the skin of the host. The bioindicator is providing also means to be able to serve as a reporter in the conditions where such cells are set to a microfluidistic chamber in the hand held device so that the analyse exposes the indicator cell via the analyte permeable membrane. Here the bioindicator regenerating cell is used in an ex vivo setting to assemble the skin structure by organ culture and organoid culture technologies that are routine in the field. Fourth the skin can be opened via the use of a small electrical current. Such a procedure has been recently shown by VTT to open up the pores of the skin and thus form a better passage for the indicator cells to enter the sweat gland cavity. Furthermore it has been shown that the electric current stimulates the skin cells to make them more receptive. The indicator cells could be mixed, for example, with a common skin cream to enable a practical way of inserting the indicator cells into the skin. Fifth, the indicator cells could be inserted into the skin by using tattoo making techniques. Sixth, the indicator cells could be inserted into the skin by using a microneedle plaster impregnated with the indicator cells with a needle height of the order of the distance between the regenerative skin cell layer and the outermost cell layer of the skin.

In one embodiment of the invention, techniques used for administering the modified cell include but are not limited to tattooing like methods (i.e. cells are administered into the skin's dermis), piercing, optical radiation, microabrasion of the skin, sticking plasters having effects on follicular orifices or hair follicles, and application of the cells on the skin. Application of the genetically modified cells on the skin of a subject allows said cells to contact with the body and function as regenerative cells. The cells obtained from a subject and furthermore genetically modified may be applied on the skin of said subject or on the skin of another subject. In one specific embodiment, the hair is removed from the hair follicle before applying the genetically modified cells on the skin.

In some embodiments of the invention, genetically modified regenerative indicator cells are administered to a subject in a non-invasive manner by applying the cells on a permeable skin area. Said skin area may have been made permeable to the cells e.g. through a cut or an abrasion, or by piercing, by puncturing, by scrubbing, by peeling, by opening hair follicles by removing one or more fine hairs therefrom (e.g. by pulling), by opening follicular orifices, hair follicles, and/or sweat gland cavities by electric current or by any other suitable means, or through any other purposively made minor skin damage or the like which opens a route for administering and incorporating the genetically modified cells into the regenerative cell layer of the skin. Permeability of the skin may also be enhanced by placing a plastic film or any other corresponding sheet-like structure on the skin, thereby increasing liquid contents of the cytoplasm of the skin cells. Also chemical means for making a skin area permeable to the present cells are envisaged. Accordingly, the genetically modified regenerative indicator cells may be administered to a subject having a permeable skin area though said skin area. Importantly, in these embodiments, treatments which make a given skin area permeable to the present cells are not part of present method. Instead, the method for obtaining indicator signals from a cell or the method for determining a biological state of a cell is practised on a subject who already has a permeable skin area.

In some other embodiments, genetically modified regenerative indicator cells may be administered to a subject through an intact skin area. This may be executed by applying the genetically modified cells topically on the intact skin. Preferably the cells are comprised in a suitable chemical composition, e.g. in the form of a cream or other carrier substance easily absorbed by the skin, to enhance incorporation of the cells into the regenerative skin layer. In other words, the skin area on which the cells are to be applied does not have to be made permeable by any pretreatment, but permeability to the cells is achieved through the composition or the carrier used for the administering the cells. Accordingly, the term “permeable skin area” also encompasses intact skin.

Amounts and regimens for application of genetically modified cells according to the present invention can be determined readily by those skilled in the art of genetically modified regenerative cells. Generally, the dosage of the genetically modified cells varies depending on considerations such as frequency of administrations (if several administrations are utilized), the type of regenerative cells and the target tissue for administration; and other variables to be adjusted by the individual physician. For instance, the regenerative cells are typically administered in an amount of a cell cluster that is composed of about 1000 cells, specifically in an amount of at least one cell given the capacity for regeneration and self-renewal. A desired dosage can be administered in one or more doses at suitable intervals to obtain the desired amount of regenerative cells in a target tissue.

The genetically modified cells may be administered to a subject only once or alternatively several times. For example, it may be desirable to apply the modified cells weekly, monthly, every six months, or yearly, depending upon the specific embodiment employed.

The genetically modified cells may be administered in any form, such as solid, semisolid or liquid form. A formulation can be selected from a group consisting of, but not limited to, solutions, emulsions, suspensions, creams, lotions, tablets and capsules. However, the genetically modified cells or the compositions comprising said genetically modified cells are not limited to a certain formulation but can be formulated into any known acceptable formulation. The compositions may be produced by any conventional processes known in the art e.g. by mixing cells and any other agent(s).

In one embodiment of the invention, before classifying a human or animal subject as a suitable target for administering genetically modified regenerative cells according to the method of the present invention, for example disease history or e.g. risk for a specific disease may be evaluated. After carrying out the method of the present invention and receiving results deviating from the normal the clinician may suggest e.g. further diagnostic methods and/or treatment for a patient.

Sensing of the present invention relies on detecting signals of indicator molecules. A signal of the indicator is monitored outside from a cell e.g. on, off or above the skin of a subject. The method of the present invention detects the presence, absence or amount of an indicator signal, which associates with the presence, absence or amount of an analyte in a cell without penetrating or breaking the cell wall. Indeed, monitoring used in the present invention is a non-surgical monitoring method or it can be converted to a surgical one where minimal cell/tissue insertion operations are needed. This is routine in the field since for example when a person has burned badly the keratinocytes are used to protect the skin for leaking and such cells then go on to recover the skin. Hence routine technologies for skin recovery exist and have been commercialized.

One advantage of the invention is that the indicator signal may be monitored in real time. This enables user friendly applications. In a specific embodiment of the invention the monitoring is carried out continuously. As used herein “continuously” refers to following up changes of the indicator signal in a non-stop way. Expression “continuously” is opposite to monitoring every now and then.

In one embodiment of the invention the monitoring is carried out by utilizing measurements selected from the group consisting of optical, conductivity, magnetic field, radiation, impedance, electrochemical, acoustic and biological measurements. Therefore, the indicator signals may be any optical (e.g. light and its reflectance, refraction, absorption or color; also change in Raman scattering properties or change in the hyperspectral fingerprint), electrical (e.g. change in skin surface electrical conductivity or surface potential), magnetic field (e.g. change in magnetic polarization, ferromagnetic resonance, electron spin resonance, electron paramagnetic resonance or nuclear magnetic resonance), radiation (e.g. changes in fluorescence resonance energy transfer, luminescence or phosphorescence), impedance (e.g. change in dielectric permittivity or its frequency spectrum), electrochemical (e.g. change in ionic conductivity or redox reaction), acoustic (e.g. change in acoustic or photoacoustic properties) and/or biological (e.g. a detectable biological change of tissue or hair) signal. The signal from the indicator molecule can be detected or monitored by various methods known in the art. These methods are well-known to a person skilled in the art and a person skilled in the art knows when and how to employ these methods. These methods include but are not limited to optical measurements such as ultraviolet, infrared, bioluminescence measurements or imaging, fluorescence measurements or imaging, measurements of radioactive labels or tracers, use of magnetic fields and/or use of X-rays or gamma radiation. The methods of the present invention may comprise transmitting to an external device outside a cell, e.g. on the skin, a signal corresponding to the presence and/or amount of one or more analytes. The cell(s) or skin of a subject is capable of being coupled to or being nearby a device adapted to detect a signal from the cell(s).

E.g. any signals of an indicator, which can be further converted to electrical signals, are suitable for the present invention. In one embodiment of the invention the indicator signal is converted to an electrical signal.

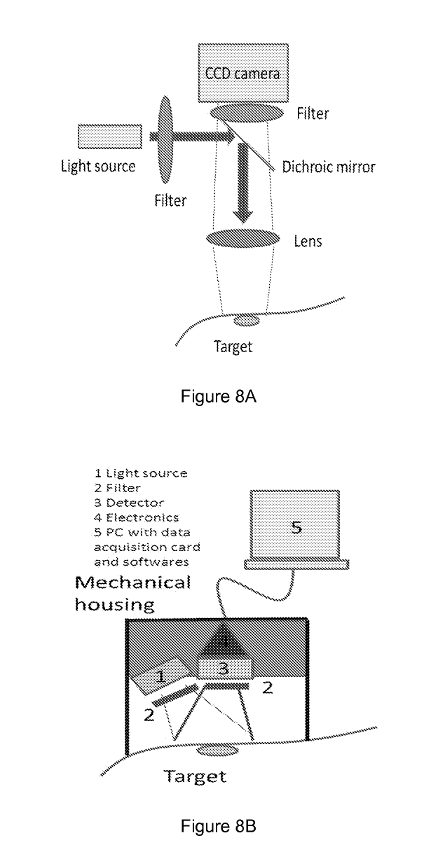

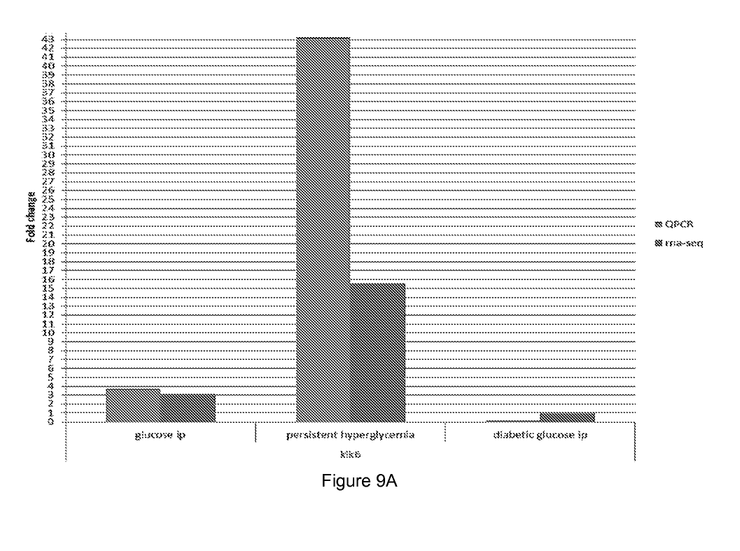

For example in one embodiment of the invention, an external light source and a fluorometer may be placed close to the modified cells for measurement of indicator signals associated with e.g. glucose using the fluorescence e.g. from the GFP or FRET pair. In another example measurement setup or device comprising a setup of FIG. 6 and/or a setup of FIG. 7 may be used in the present invention. In one embodiment of the invention, the measurement is performed by using a low-cost LED for illuminating the skin underneath a wrist device and then reading the indicator signal using a low-cost photodetector. Both the LED and the photodetector may be placed on the bottom surface of the wrist device in such a way that the ambient light does not disturb the actual measurement.

Monitoring step of the present invention detects the presence, absence or amount of an analyte in a cell by non-surgical or non-invasive means. By “non-surgical” or “non-invasive” it is meant that no break in the skin is created and monitoring is not carried out inside a subject or cell.

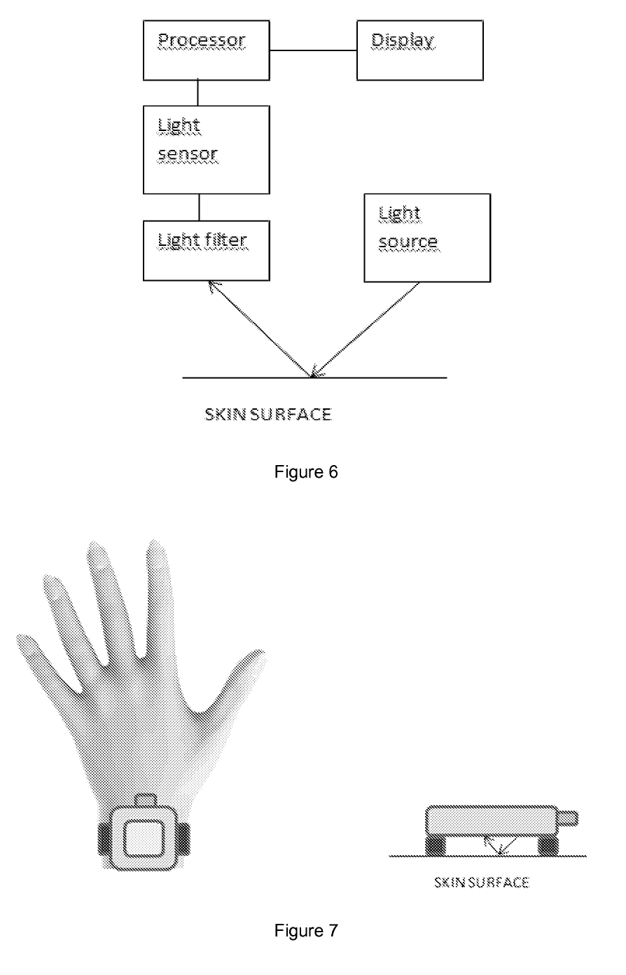

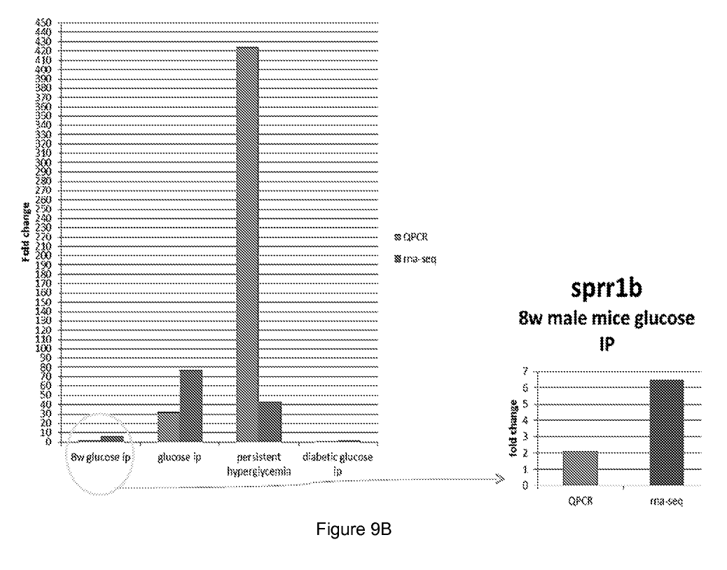

In one embodiment of the invention the method further comprises a step of converting the indicator signal to a value, quantitative or qualitative value, numerical value, result revealing a trend or on/off result. In a specific embodiment of the invention, a device adapted to detect a signal from the cell is a reader device or comprises a reader device with the electronics and/or signal processors that are needed for the display of the results in a user-friendly way. Monitoring set ups or devices suitable for the present invention include but are not limited to those described or shown e.g. in FIGS. 5B-8.

The invention also relates to a system comprising means for modifying the regenerative cell by inserting a polynucleotide sequence encoding an indicator into DNA of the regenerative cell, the polynucleotide sequence encoding the indicator to be expressed together with a target polynucleotide in said regenerative cell; and means for monitoring a signal of the indicator or absence thereof on the skin of the subject. In one embodiment the system further comprises means for administering the modified cells on the skin of a subject or into a subject. In one embodiment of the invention the system also comprises the regenerative cells to be modified.

In a specific embodiment of the invention the system is for carrying out the method of the present invention.

As used herein “means for modifying the regenerative cell” refer to any devices and/or agents for modifying regenerative cells and as an example may be selected from the group consisting of a kit for modifying regenerative cells, reagents (e.g. buffer) necessary for performing the modification, suitable primers or probes, polynucleotide(s) encoding and indicator(s), and devices such as pipettes or vials suitable for modifying the cells. Methods for modifying the regenerative cells have been described earlier in the disclosure and belong to general knowledge of a man skilled in the art.

As used herein “means for monitoring a signal of the indicator or absence thereof on the skin of the subject” refer to any devices, apparatus or set ups for monitoring indicator signals on the skin of the subject. Examples of suitable devices, apparatus or set ups have been described earlier in the disclosure and are within general knowledge of a man skilled in the art.

As used herein “means for administering the modified cells on the skin of a subject or into a subject refer to any devices and/or agents used for applying the cells, and may be selected e.g. from the group consisting of needles, syringes, pipettes, vials, lotions, creams, liquids, and any other agents such as acceptable carriers, buffers, excipients, adjuvants, additives, antiseptics, filling, stabilising and/or thickening agents, and/or any components normally found in corresponding products. Selection of suitable ingredients and appropriate means belongs to general knowledge of a man skilled in the art.

It will be obvious to a person skilled in the art that, as the technology advances, the inventive concept can be implemented in various ways. The invention and its embodiments are not limited to the examples described above but may vary within the scope of the claims.

### EXAMPLES

### Example 1

### Identification of Genes to be Used as Markers

In one example of the invention glucose responsive genes were screened from mouse skin:

In vivo glucose response in skin was determined using mice. Animal were fasting for 12 hours, then they were weighted and anesthetized using ratanest and divided in 2 groups. The experimental animals were injected intra-peritoneally with 2 g/kg D-glucose in solution and controls with our D-glucose on the buffer. Blood glucose was measured in all animals using Bayer Contour® glucose meter with strips. After 45 minutes, blood glucose had passed the maximum and animals were killed by cervical dislocation, back skin was shaven, disinfected with 70% EtOH and skin samples were cut and snap frozen in liquid N₂. RNA was extracted with Trizol™ (Thermofisher, Vantaa, Finland), frozen skin was dissociated in Tissulyzer™ (Qiagen, Helsinki, Finland) between two 7 mm metal beads for 5 minutes, after beads removal, skin lysates were centrifuged for 10 minutes at 10000 g at 4° C. and supernatant was mixed with 0.2 volume of CHCl₃, mixed for 15 seconds, incubated at room temperature and centrifuged for 18 minutes at 12000 g. Then procedure continued as recommended by the manufacturer. After measurement of concentration and canalizing of RNA integrity on Qiaxel (Qiagen, Helsinki, Finland), 12 μg of total RNA were precipitated with 1/10 volume of Na-acetate pH 5.3 and 2.5 volume 100% EtOH and resuspended in H₂O, polyA RNA were selected using Poly(A)Purist™ (Ambion, Austin, USA) according to the manufacturer's instructions. Libraries were made from polyA-RNA from 6 samples (3 from baseline and 3 from glucose-injected animals) and sequenced. Comparison of reads between control and glucose injected animals were done. Genes which, had at least 2 fold change (+or −) and p value<0.1 were considered to be sensitive to glucose. QPCR to verify this was done on samples from other animals than those used for RNA-seq.

Time of harvesting RNA was determined by blood test showing response to stimuli (depending on the experiment stimuli could be: response to cancer-related molecules, prion protein, amyloid protein, any pathology related molecule, which can be detected, from blood or body fluid) and compared to healthy/unstimulated control at same time.

Genes, which calculated fold change equal or superior to 2 was considered a candidate.

Simultaneous significant changes of a number of genes identified by this method were considered a signature of the stimulus.

Candidates were selected on 2 bases:

1: responding to stimulus in both young and older animals (5 controls and 5 treated), considered transient response as up or down-regulated

2a: responding to stimulus in both young and older animals by up-regulation (longer term response not going down as soon as glucose is cleared from blood)

2b: responding to stimulus by up-regulation and remaining up-regulated in high blood glycemia (not enough insulin for clearing) Transiently changed genes are shown in Table 1.

TABLE 1
Transiently changed genes.

| | | |
|---|---|---|
| Lce1d\|2310037L11Rik\|AI415320\|SprrI7\|-\|3 F1\|3\|protein- | −1.655531319 | 1.465571559 |
| coding | | |
| Lce1e\|1110031B11Rik\|AI507275\|-\|3 F1\|3\|protein-coding | −1.510202995 | 1.440924645 |
| Lce1gI1110058A15Rik\|AI604448\|-I3 F1\|3\|protein-coding | −1.585360952 | 1.448005878 |
| Lce1h\|2310066F03Rik\|AI426284\|SprrI9\|-\|3 F1\|3\|protein- | −2.393933093 | 1.693534178 |
| coding | | |
| Lce1k\|Gm7055\|SprrI6\|mCG_1042747\|3 F1\|3\|protein- | −1.72294264 | 1.415076527 |
| coding | | |
| Lef1\|3000002805\|AI451430\|Lef-1\|-\|3 G3\|3 60.78 | −3.092714404 | 2.261416171 |
| cM\|protein-coding | | |
| Gm17757\|-\|mCG_147564\|7\|7\|pseudo | −1.912901942 | 2.261919765 |
| Ctss\|-\|-\|3 F2.1\|3 40.74 cM\|protein-coding | −1.816654245 | 1.849221398 |
| Fcgr1\|AI323638\|AV092959\|CD64\|FcgammaRI\| | −2.376392567 | 2.442963886 |
| IGGHAFC\|-\|3 F2.1\|3 41.72cM\|protein-coding | | |
| Adamts18\|9630038L21\|ADAMTS21\|E130314N14Rik\|-\| | 2.886641599 | −3.132621005 |
| 8 E1\|8\|protein-coding | | |
| Bcl2a1a\|A1\|BB218357\|Bcl2a1\|Bfl-1\|Hbpa1\|-\|9 E3.1\|9 | −2.411637191 | 1.985810949 |
| 47.24 cM\|protein-coding | | |
| Bcl2a1b\|A1-b\|-\|9 E3.1\|9\|protein-coding | −2.327708907 | 2.181433741 |
| Bcl2a1d\|A1-dI-I9 E3.1\|9\|protein-coding | −2.538595093 | 2.13693688 |
| Ccl4\|AT744.1\|Act-2\|MIP-1B\|Mip1b\|Scya4\|RP23- | −6.092636266 | 4.016800421 |
| 320E6.8\|11 C\|11 51.09 cM\|protein-coding | | |
| Cc\|7\|MCP-3\|Scya7\|fic\|marc\|mcp3\|RP23-350G1.4\|11 | −2.471401372 | 2.273775676 |
| C\|11 49.83 cM\|protein-coding | | |
| Ccr1\|Cmkbr1\|Mip-1a-R\|-\|9 F\|9 75.05 cM\|protein-coding | −3.066525723 | 3.41299971 |
| Ccr5\|AM4-7\|CD195\|Cmkbr5\|-\|9 F\|9 75.05 cM\|protein- | −2.775624865 | 2.110133423 |
| coding | | |
| Clec4d\|Clecsf8\|Mpcl\|mcl\|-\|6 F3\|6 58.33 cM\|protein- | −2.95989187 | 3.430918551 |
| coding | | |
| Klk9\|1200016C12Rik\|AI324041\|-\|7 B4\|7 28.26 cM\|protein- | −1.631509774 | 1.307721607 |
| coding | | |
| Ifi47\|47 kDa\|IRG-47\|Ifggc1\|Igrd\|Iigp4\|Iipg4\|Irgd\|RP23- | −1.912426029 | 1.61110069 |
| 54N20.3\|11 B1.2\|11\|protein-coding | | |
| Iigp1\|2900074L10Rik\|AI046432\|AW111922\|Ifgga1\|Iigp\| | −1.943763104 | 1.6024054 |
| Irga6\|-\|18 D3\|18\|protein-coding | | |
| Il1rl1\|DER4\|Fit-1\|Ly84\|ST2L\|St2\|St2-rs1\|T1\|T1/ST2\|-\|1 | −4.327579429 | 2.694007767 |
| B\|1 19.19 cM\|protein-coding | | |
| Pyhin1\|4930422C14\|AI447904\|lfi209\|lfix\|-\|1 H3\|1\|protein- | −2.689394955 | 2.077118521 |
| coding | | |
| Slfn2\|Shlf2\|RP23-392I13.11-001\|11 C\|11 50.3 cM\|protein- | −1.682555392 | 1.61257755 |
| coding | | |
| Slfn3\|-\|RP23-381B19.1\|11 C\|11\|protein-coding | −2.38699284 | 2.64097503 |

TABLE 2
Longer-term changed genes.

| | | |
|---|---|---|
| Klk6\|AI849898\|BSP\|Bssp\|Klk29\|MSP\|Prss18\|Prss9\|neurosin\|-\| | 1.61549065 | 2.006481579 |
| 7 B4-B5\|7 28.28 cM\|protein-coding | | |
| Sprr1b\|-\|-\|3F1\|\|3 40.14 cM\|protein-coding | 6.275434797 | 2.694833138 |
| Stfa2\|Stf2\|-\|16 B3\|16 25.52 cm\|protein-coding | 3.895992442 | 2.585302956 |

For example any of the glucose responsive genes of Table 1, 2 or 3, or in FIG. 9 or 10 or any combination thereof may be utilized in the present invention.

The above mentioned assay is routine also in humans when the functionality of the pancreas in producing insulin is tested for example.

In another set of experiments aimed at identifying proteins which are up-regulated after glucose injection, both healthy and type I diabetic mice were employed. To this end, type 1 diabetes was induced in a subset of mice by a single intravenous injection (under anesthesia) of 150 mg/kg streptozotocin (STZ) in citrate buffer (pH 4.5) at the age of 4-8 weeks. Blood glucose was monitored every 48 h, and one week after the injection all STZ-treated mice were diabetic. The mice were then divided into four study groups (n=11) as follows:

G1: Healthy mice (Control, Ctrl) injected with water

G2: Healthy mice (Control, Ctrl) injected with glucose

G3: Type I diabetic mice injected with water

G4: Type I diabetic mice injected with glucose

Proteins were extracted from six skin samples from each study group and subjected to proteomics analysis. To this end, a piece of skin was cut and put in a pre-cooled tube containing two 5 mm metal beads. The skin was lyzed using 1 ml of lysis buffer disclosed below for 5 minutes at 50 Hz in TissueLyser (Qiagen). A thick unbreakable and insoluble membranous piece left-over was removed. Then the samples were spun for 15 minutes at 13000 rpm at 4° C. and the supernatant was put in a fresh tube and stored at −70° C. Protein concentrations were measured using a Pierce BCA kit (Thermo Fisher) in accordance with the manufacturer's instructions. Equal amounts of total protein (50 μg) from control mice were labeled with Cy3 (minimal DIGE) and separated by IEF (pH4-7) and SDS-PAGE. Changes in protein spot positions were detected, and then the expression profiles of each protein were quantified and analyzed statistically. Protein expressions were considered as significantly decreased or increased when the p value was <0.05.

### Lysis Buffer

25 mM HEPES

0.3 M NaCl,

1.5 mM MgCl₂,

0.2 mM EDTA

0.1% Triton X-100,

0.5 mM dithiothreitol,

1 Tablet of protease inhibitor (Sigma) for 100 μl final volume.

2 Tablets of phosphatase inhibitor (Sigma, Mirja stock) for 100 μl final volume

The results showed that three proteins were significantly increased (p<0.05) after glucose injection both in healthy and type I diabetic mice. These proteins and relative expression ratios thereof between different study groups are shown in Table 3 below:

TABLE 3

| | Ratio | | | | | T-test | | | | |
|---|---|---|---|---|---|---|---|---|---|---|
| Protein | G2/G1 | G3/G1 | G4/G1 | G4/G2 | G4/G3 | G2/G1 | G3/G1 | G4/G1 | G4/G2 | G4/G3 |
| Alpha-2-HS- | 0.80 | 1.51 | 1.17 | 1.46 | 0.78 | 0.03 | 0.00 | 0.18 | 0.02 | 0.05 |
| glycoprotein | | | | | | | | | | |
| Protein | 1.32 | 1.45 | 2.19 | 1.66 | 1.51 | 0.01 | 0.09 | 0.00 | 0.00 | 0.02 |
| LYRIC | | | | | | | | | | |

Polynucleotides encoding the identified glucose-responsive proteins are non-limiting examples of suitable target polynucleotides to be expressed together with a polynucleotide encoding an indicator.

In a further example of the invention, glucose responsive genes were screened from skin progenitor cells:

Skin progenitor cells (SKPs) were isolated from normal human skin of patients undergoing plastic surgery as previously described (Rezvani HR et al. J Clin Invest. 2011 January; 121(1):195-211). Briefly, fresh skin fragments were immediately cut into 5×5 mm pieces and treated with trypsin for 3 hours at 37° C. or overnight at 4° C. to separate the epidermis from the dermis. SKPs were seeded at a concentration of 10⁵ cells/cm² in Keratinocyte-SFM (1×) medium, supplemented with hydrocortisone (0.5 mg/ml), epidermal growth factor (10 ng/ml), insulin (5 mg/ml). The medium was changed three times a week. When the cultures reached 70-80% confluence, the cells were detached with 10% trypsin and then resuspended in Keratinocyte-SFM (1×) medium to be used for transduction experiment (e.g. as described in example 3) or were used for glucose treatment (e.g. as described in example 1 or 2), and/or were used for transplantation experiment (e.g. as described in example 4).

To find out glucose responsive genes, SKPs were treated with two concentration of glucose (6 and 26 mM). RNA samples were collected at different end points (5-45-90 min and 6 h, 24 h). RNA extraction and RNA-Seq were performed as described above under glucose responsive genes in relation to mouse skin.

According to the results for example any of the glucose responsive genes of Table 1, 2 or 3, or in FIG. 9 or 10 or any combination thereof may be utilized in the present invention.

### Example 2

### Isolating Skin Stem Cells from Mouse Skin

Skin stem cells were isolated from skin of 6-8 week old mice. The mice were sacrificed (by cervical dislocation) then the hair was shaved. The skin was sterilized by immersion in beaker with 10% betadine for 2 min, in beaker with 70% ethanol for 1 min and then in sterile PBS for 1 min. Skin samples were treated with trypsin overnight at 4° C. Epidermis was separated from dermis, minced and transferred into sterile 50 ml falcon tube containing trypsin. Epidermal fractions were filtered through 40 mm filter and cell pellet was suspended in PBS with 0.5% BSA and then stained with CD34 and CD49f (α6-integrin) antibodies for 1 hour on ice. Staining analysis was performed using a flow cytometer (FACS). CD49f and CD34 positive cells were selected and plated in FAD-DMEM medium

The cells were co-cultured with a feeder (3T3 cells which are fibroblast cell line). These last cells were providing the skin stem cells all the adhesion molecules which are necessary for the growth of the stem cells. However, after some expedient we realized that the co-culture of stem cells and 3T3 cells affected the normal proliferation of skin stem cells. We decided to cultivate the skin stem cells without any feeder and we were able to prepare different skin stem cells from different mouse individuals. In addition, we were able to keep the stem skin cells till passage 2. This technology was based similarly on isolating the skin cells via biopsy, dissociating the cells by means of mild enzyme treatment and the using FACS to purify with an antibody marker set the stem cell pool of the cells and the other skin constituent cells. Such cells were placed to cell culture and the gene editing was done there in. When the technologies will become available the targeted gene editing is aimed also to be in vivo by using the skin stem cell targeting exosomes.

The different types of cells were treated with two concentration of glucose (6 and 26 mM) according to routine glucose challenge, tolerance protocols. RNA samples have been collected from these cells at different end points (5-45-90 min and 6 h, 24 h) according to commercial kits and RNA extraction, RNA sequencing and QPCR were performed as described in example 1 to find out glucose responsive genes (see Tables 1, 2 and 3, and FIGS. 9 and 10).

### Example 3

### Inserting an Indicator Polynucleotide Sequence into Cell DNA

Any known indicator or indicators may be used in the present invention. Suitable indicators are well-known to a person skilled in the art and a person skilled in the art knows when and how to employ these indicators. Examples of suitable indicators include but are not limited to fluorescent proteins, a green fluorescence protein (GFP), GFP derivative, photoprotein (e.g. firefly luciferin protein), mCherry, yellow fluorescent protein, tomato red protein, lusiferase reporter, FRET donor and/or acceptor protein, aptamer polynucleotide and/or aptamer polypeptide, myc tag, flag tag, halo tag, biotin/avidin tags and their modifications, unnatural bases and transfer RNA and amino acid based tagging, the polypeptides that serve as electricity indicators and those genes encode for the pigments of body such as the melanin and eye color pigments. In one embodiment, the indicator is GFP.

Any known methods and means may be used in the present invention for inserting an indicator polynucleotide into target DNA. Suitable methods and means are well-known to a person skilled in the art and a person skilled in the art knows when and how to employ these methods and means. Examples of suitable methods include but are not limited to those utilizing Zinc finger nucleases, Transcription Activator-Like Effector Nucleases (TALENs), the CRISPR/Cas system or engineered meganuclease re-engineered homing endonucleases.

In one embodiment, an indicator (such as GFP) polynucleotide was inserted into a gene identified as glucose responsive in the skin and mentioned e.g. in the Table 1, 2 or 3, or FIG. 9 or 10, or close to said gene in order to be expressed together with said gene. According to FIG. 9 Kalrein6, Sprr1 and Pyhin1 gene expressions changed dramatically upon modification of the availability of glucose. These gene sequences were derived from the public databanks (NIH, EMBL). A crisp/cas technology based construct, thus the guide RNA for each of these genes are designed and obtained from commercial sources. By using already routine and commercially available technologies the guide RNAs and the cas are introduced to the skin regenerating cell of a human or animal, e.g. to the cell of Example 2. The insertion of nucleotide sequence of GFP to target gene Kalkrein 6, Sprr1b and Pyhin 1 (or close to it) is carried out according to the method described in the article of Sander and Joung (2014, Nature Bio-technology 32, 347-355). The gene editing generated by the designated guide RNAs is confirmed by sequencing, PCR and Southern Blotting. The off targets are also analyzed by from the genome wide sequence data from the gene edited cells. Once this has been completed the gene edited cell is subjected to testing of the functionality of the indicator. For example GFP indeed behaves as expected. The regenerating cell is exposed to glucose and insulin and the changes in the indicator are read by confocal microscope based in the emitted energy from the GFP. Once this has been confirmed the reporter cell containing the indicator in a defined, designed gene locus depicted in this invention description is grafted in vivo to a nude mice by the technologies described in this invention disclosure. The functionality of the regenerating reporter carrying cells is then exposed in vivo to the glucose tolerance test. This part of the process serves to confirm the proper functionality of the indicator in vivo. After this step in bioreporter generation process the cell is transferred as ectopic subject skin pore present cell or as an integrant to the skin layer that goes to regenerate the skin as outlined in this disclosure.

In one embodiment of the invention indicator polynucleotide sequences were inserted into cell DNA as described below in Example 3.

### CRISPR Lentiviral Particles and Skin Progenitor Cell Transduction

RNA-Seq data of example 1 or 2 showed that one gene of kallikrein subfamily members: KLK6 (kallikrein related peptidase 6) was significantly up regulated after glucose injection in vivo (in mice) and in vitro (progenitor cells). To knockout the expression KKL6 in SKPs, CRISPR lentiviral particles were constructed based on three gRNA sequences: AAGCATAACCTTCGG-CAAAGGG (SEQ ID NO: 1), GAGCAGAGTTCTGTTGTCCGGG (SEQ ID NO: 2), and CCCTGACTATGATGCCGCCAGC (SEQ ID NO: 3) (Sigma-Aldrich, Finland). The lentiviral particles contained two selection markers (GFP and Puro), which provide multiple options for monitoring the cell population.

For transduction, human skin progenitor cells (5×10⁵ cells per T25 flasks) of example 1 were incubated for 24 hours in complete medium. Prior to infection, the medium was removed, and the cells were incubated with viral supernatants for 24 hours at 37° C. in the presence of 8 μg/ml of protamine sulphate. After 5 days, the transduction efficiency was determined by Flow cytometer (FACS) based on the percentage of GFP-positive cells.

### Western Blotting Procedure to Confirm Gene Editing

To verify the efficiency of KLK6 knockout, western blotting was performed as previously described (Rezvani HR et al. J Clin Invest. 2011 January; 121(1):195-211). Briefly, equal amounts of total protein was resolved by SDS-PAGE and electrophoretically transferred to PVDF membranes. The membranes were then incubated overnight at 4° C. with a 1:200 dilution of the anti-KLK6 (clone 4A10, Sigma-Aldrich, Finland), and 1:1000 dilution of anti-β-actin antibody (ab8227, abcam). After additional incubation with a 1:10,000 dilution of an anti-immunoglobin horseradish-peroxidase-linked antibody (Vector Laboratories) for 1 hour, blots were developed using the chemiluminescence ECL reagent (Amersham Biosciences, Finland).

The method used for the depletion of KLK6 gene in the skin progenitor cells with the CRISPR/Cas9 is utilized for overexpressing KLK6 fusion with the GFP sensor. The modified cells overexpressing KLK6 fusion with the GFP sensor are then injected to a mice or a human subject or placed on the skin thereof.

### Example 4

### Development of Technology to Integrate Skin Cells to a Recipient

Transplantation experiments were performed in order to test the integration of GFP⁺ cells (from GFP mouse) into WT recipient mouse. Two different methods (A and/or B) were used. For example either A or B or both A and B can be utilized in the method of the present invention. Also, similar results are obtained by applying the genetically modified cells or a composition comprising said cells on top of the skin of a subject (e.g. a mouse or human subject) when the modified cells enter the hair follicle or sweat gland.

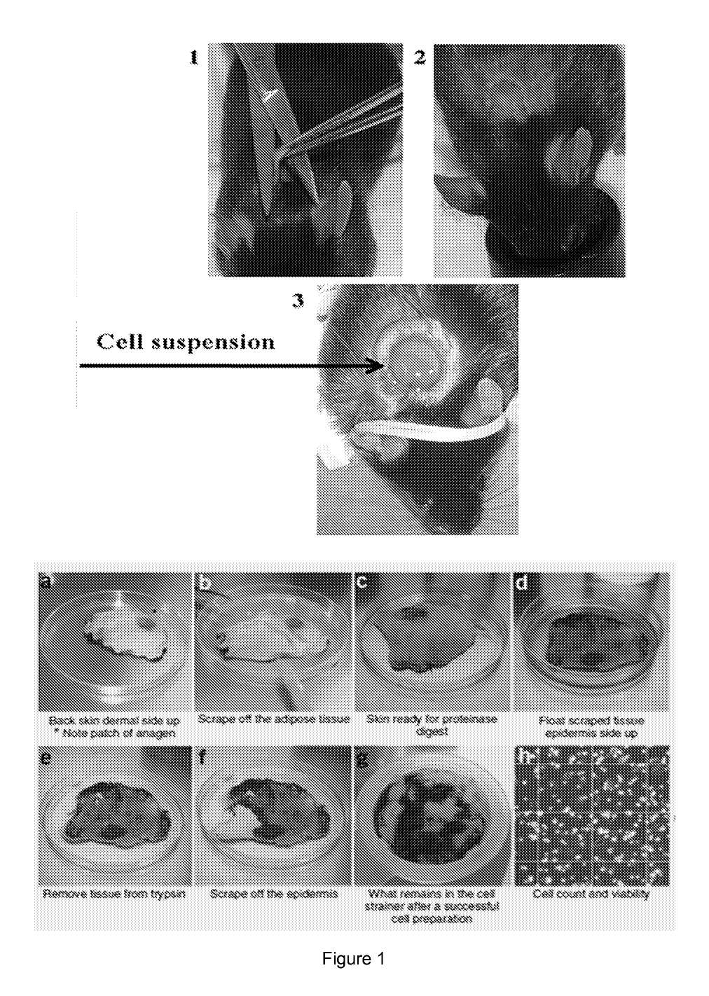

A. Xenograft of Skin Stem Cells Using Silicon Chamber (FIG. 1).

The GFP positive cells were prepared as described above, 2×10⁶ cells were injected in the silicon chamber. The results showed that the donor cells were able to integrate (proliferate and migrate) nicely into recipient skin mouse and stay for at least for 2 months. These results were confirmed by GFP antibody immuno-staining.

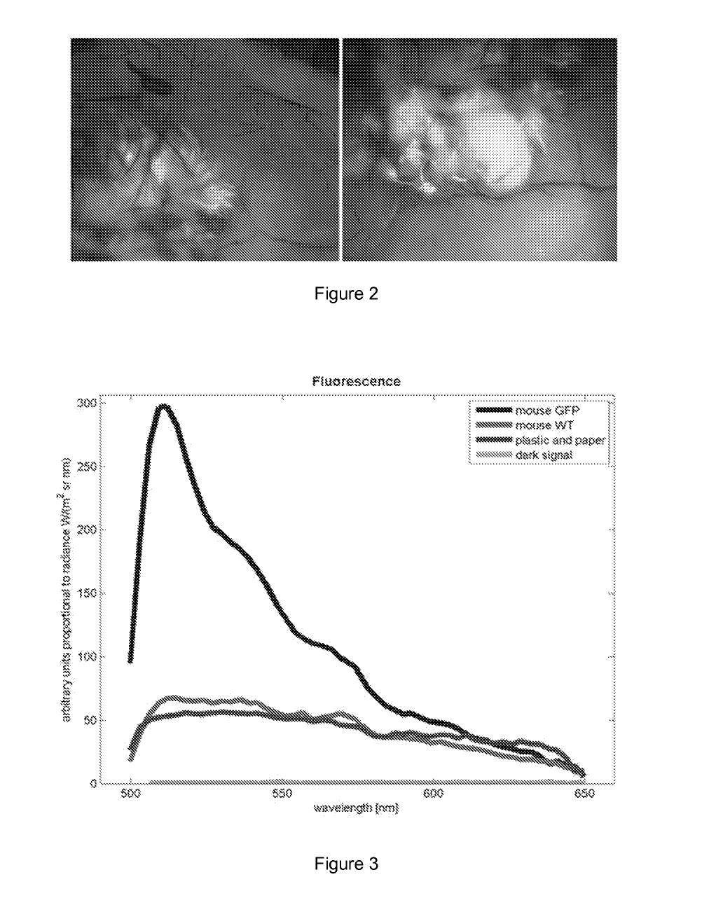

B. GFP Skin Graft (the Whole Skin) on the Back of WT Recipient Mouse (FIG. 2).

In this protocol we cut the whole skin of GFP mouse in small pieces (chopped skin). These pieces were injected into a small pocket of the WT mouse. The integration of the GFP skin was detected up of two months by fluorescent microscopy and then confirmed by the GFP antibody immune-staining.

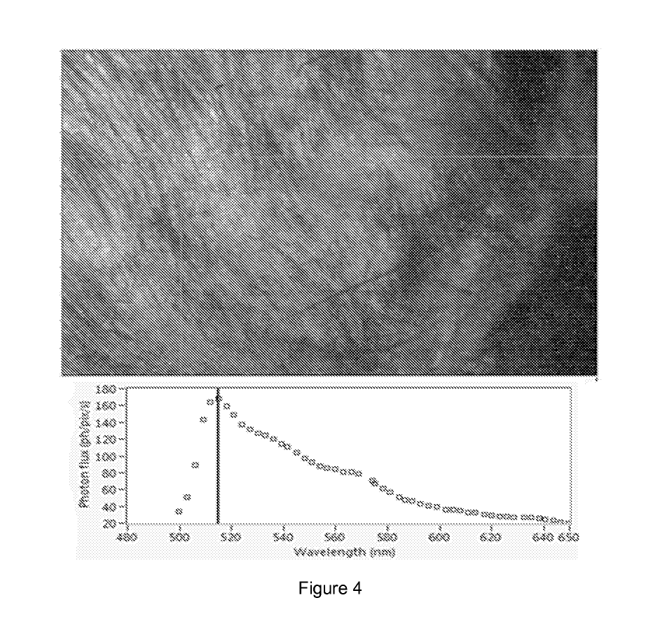

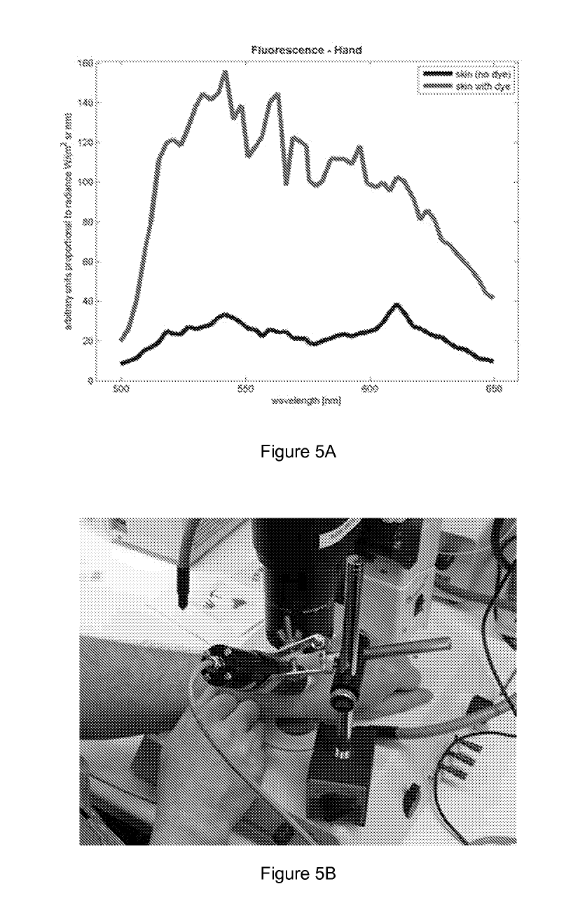

FIGS. 3-4 reveal the results of transplantation experiments. FIG. 5A reveals that the method of the present invention works and that the indicator signals can be obtained from skin by using the method of the present invention.

A further transplantation experiment was performed in order to demonstrate that modified regenerative cells can be transplanted successfully in a non-invasive manner by topical administration to a skin area containing a skin lesion. For this experiment, GFP-positive mice in which the fluorescent GFP protein is constitutively expressed in all organs, and notably in skin, were used as donors. C57BL/6 mice which are black without any fluorescent protein expression were employed as recipients. The C57BL/6 mice were euthanized with CO₂ and the fur from the back was pulled-off using forceps. Once the fur was removed, the whole skin from the back was collected and cut in different pieces. A silicon chamber was installed on the top of each piece. GFP-positive stem cells were freshly isolated from GFP mice as describe above, and applied into the silicon chamber. The GFP signal was traced after 24 or 72 h, and pictures were taken to show the ability of these cells to integrate in the hair follicle.

The outcome of this experiment indicates that removal of skin hairs can be used as a route for integrating genetically modified indicator cells in the skin without any injection under the skin just by applying them on the top of the skin e.g. as a cream.

In one embodiment of the invention human skin progenitor cells are implanted in vivo to a mouse. SKPs culture is performed as described above in example 1. These cells are transduced with lentiviral particles CRISPR to over-express KLK6 GFP fusion. FACS based on the percentage of GFP-positive cells is utilized for determining the efficiency of transduction.

A small silicon chamber is inserted into skin mouse and GFP positive cells are injected. 24 h post-injection, a small hall is applied followed by removing the silicon chamber one week after the injection. The integrity of the GFP positive cells is checked using a fluorescent microscope (one week, two weeks, one month and, two months).

The recipient mice of GFP positive cells and control mice are treated with different concentrations of glucose. Then the expression of KLK6 is traced following the GFP signal showing that this gene is a specific glucose biosensor.

Transplantation experiments are also performed in a human. The edited cells are applied on the skin of a person or administered to the basal cell layer or epidermis of the skin that is able to renew the skin. Alternatively, tattooing like methods, piercing, optical radiation, micro-abrasion of the skin, sticking plasters having effects on follicular orifices or hair follicles are utilized.

### Example 5

### Monitoring Biochemical Changes of Cells

Any known monitoring methods and means may be used in the present invention for monitoring the indicator signal of cells associated with expression of a specific gene. These methods are well-known to a person skilled in the art and a person skilled in the art knows when and how to employ these methods. Examples of suitable monitoring methods include but are not limited to optical measurements such as ultraviolet, infrared, bioluminescence measurements or imaging, fluorescence measurements or imaging, measurements of radioactive labels or tracers, use of magnetic fields and/or use of X-rays or gamma radiation.

In one embodiment the monitoring was carried out by utilizing optoelectronic methods or means (e.g. hyperspectral methods and/or cameras). Monitoring set ups or devices suitable for the present invention include but are not limited to those described or shown e.g. in FIGS. 5B-8.

1. A method for obtaining indicator signals from a cell, said method comprising
providing regenerative cells obtained from a subject,
modifying the regenerative cell by inserting a polynucleotide sequence encoding an indicator into DNA of the regenerative cell, the polynucleotide sequence encoding the indicator to be expressed together with a target polynucleotide in said regenerative cell,
administering the modified regenerative cell to a subject,
monitoring a signal of the indicator or absence thereof on the skin of the subject, thereby obtaining indicator signals associated with expression of the target polynucleotide.

2. A method for determining a biological state of a cell, said method comprising
providing regenerative cells obtained from a subject,
modifying the regenerative cell by inserting a polynucleotide sequence encoding an indicator into DNA of a regenerative cell, the polynucleotide sequence encoding the indicator to be expressed together with a target polynucleotide in said regenerative cell,
administering the modified regenerative cell to a subject,
monitoring a signal of the indicator associated with expression of the target polynucleotide or absence of said indicator signal on the skin of the subject, and
determining a biological state of the cell defined by expression of the target polynucleotide.

3. The method according to claim 1, wherein the subject has a permeable skin area and the modified regenerative cells are administered in a non-invasive manner by topical application on said permeable skin area.

4. The method according to claim 1, wherein modified regenerative cells are administered to a subject by a method selected from the group consisting of tattooing like methods, piercing, optical radiation, micro-abrasion of the skin or application of the cells on the skin of a subject.

5. The method according to claim 1, wherein the indicator signal is converted to an electrical signal.

6. The method according to claim 1, wherein the monitoring is carried out continuously.

7. The method according to claim 1, wherein the monitoring is carried out by utilizing measurements selected from the group consisting of optical, conductivity, magnetic field, radiation, impedance, electrochemical, acoustic or biological measurements.

8. The method according to claim 1, wherein the method further comprises a step of converting the indicator signal to a value, quantitative or qualitative value, numerical value, result revealing a trend or on/off result.

9. The method according to claim 1, wherein the method further comprises a step of culturing the regenerative cells.

10. The method according to claim 1, wherein the indicator is selected from the group consisting of fluorescent proteins, a green fluorescence protein (GFP), GFP derivative, photoprotein (e.g. firefly luciferin protein), mCherry, yellow fluorescent protein, tomato red protein, lusiferase reporter, FRET donor and/or acceptor protein, aptamer polynucleotide and/or aptamer polypeptide, myc tag, flag tag, halo tag, biotin/avidin tags and their modifications, unnatural bases and transfer RNA and amino acid based tagging, the polypeptides that serve as electricity indicators and those genes encoding for the pigments of body such as the melanin and eye color pigments.

11. The method according to claim 1, wherein insertion of a polynucleotide sequence encoding the indicator is carried out by using site-specific nucleases.

12. The method according to claim 1, wherein insertion of a polynucleotide sequence encoding the indicator is carried out by zinc finger nuclease (ZFN), transcription activation-like effector nuclease (TALEN) mediated genome editing or CRISPR/Cas system.

13. The method according to claim 1, wherein the expression of the target polynucleotide is affected by an analyte.

14. The method according to claim 1, wherein the target polynucleotide is selected from the group consisting of polynucleotides encoding glucose responsive polypeptides, growth factors, mitochondrial enzymes, hormone responsive polypeptides, stress responsive polypeptides, polypeptides of the central or peripheral nervous system function, alcohol or drug responsive polypeptides, polypeptides used in immunological monitoring of disease development, polypeptides revealing changes in physical forces such as pressure or stretching, polypeptides expressed by physical load in exercise or pathogen infections, any polypeptide presented in the list of Table 1, Table 2, Table 3, FIG. 9 or FIG. 10, and any combination thereof.

15. Use of a regenerative cell obtained from a subject and modified to comprise an inserted polynucleotide sequence of an indicator to be expressed together with a target polynucleotide for monitoring an indicator signal associated with expression of the target polynucleotide, wherein monitoring is carried out on the skin of the subject.

16. A regenerative cell obtained from a subject and comprising an inserted polynucleotide sequence encoding an indicator to be expressed together with a target polynucleotide.

17. The cell according to claim 16 further comprising an indicator signal.

18. The cell according to claim 16, wherein the indicator polynucleotide is GFP and the target gene is selected from genes listed in Table 1, Table 2, Table 3, FIG. 9 or FIG. 10.

19. The cell according to claim 16, wherein the cell is for measuring the indicator signal associated with expression of the target polynucleotide on the skin of a subject.

20. Use of a polynucleotide encoding an indicator, which polynucleotide is inserted into a target polynucleotide in a regenerative cell and which polynucleotide is to be expressed together with the target polynucleotide, for determining an analyte of the cell, wherein the analyte is able to control expression of the target polynucleotide.

21. The method according to claim 1, wherein the regenerative cell is selected from the group consisting of skin derived regenerative cells, blood derived regenerative cells and iPS cells.

22. A system comprising means for modifying the regenerative cell by inserting a polynucleotide sequence encoding an indicator into DNA of the regenerative cell, the polynucleotide sequence encoding the indicator to be expressed together with a target polynucleotide in said regenerative cell, means for applying the modified cells on the skin of a subject or into a subject, and means for monitoring a signal of the indicator or absence thereof on the skin of the subject.

23. The system according to claim 22 for carrying out a method for obtaining indicator signals from a cell, said method comprising
providing regenerative cells obtained from a subject,
modifying the regenerative cell by inserting a polynucleotide sequence encoding an indicator into DNA of the regenerative cell, the polynucleotide sequence encoding the indicator to be expressed together with a target polynucleotide in said regenerative cell,
administering the modified regenerative cell to a subject,
monitoring a signal of the indicator or absence thereof on the skin of the subject, thereby obtaining indicator signals associated with expression of the target polynucleotide.
